(19) United States
(12) Patent Application Publication
(10) Pub. No.: US 2013/0245451 A1
(43) Pub. Date: Sep. 19, 2013

(54) ULTRASONIC PROBE AND ULTRASOUND DIAGNOSTIC APPARATUS

(71) Applicants: KABUSHIKI KAISHA TOSHIBA, Tokyo (JP); TOSHIBA MEDICAL SYSTEMS CORPORATION, Otawara-shi (JP)

(72) Inventors: Fumio Mochizuki, Nasushiobara-shi (JP); Tatsuo Ogasawara, Otawara-shi (JP); Kazuhito Nakata, Otawara-shi (JP); Kenichi Unayama, Otawara-shi (JP); Gen Nagano, Nasushiobara-shi (JP); Takeshi Fukasawa, Nasushiobara-shi (JP); Koji Kuroiwa, Nasushiobara-shi (JP)

(73) Assignees: Toshiba Medical Systems Corporation, Otawara-shi (JP); Kabushiki Kaisha Toshiba, Tokyo (JP)

(21) Appl. No.: 13/800,514

(22) Filed: Mar. 13, 2013

(30) Foreign Application Priority Data
Mar. 13, 2012 (JP) 2012-056120
Feb. 18, 2013 (JP) 2013-029116

(51) Int. Cl. A61B 8/00

(52) U.S. Cl. CPC A61B 8/4444
USPC 600/459

(57) ABSTRACT

An ultrasonic probe according to an embodiment includes a receiving unit and a light emitting unit. The receiving unit receives input operation related to transmission and reception of ultrasonic waves performed by a piezoelectric vibrator transmitting and receiving ultrasonic waves under the control of an apparatus body. The light emitting unit emits light in a first light emission state when connected to the apparatus body and emits light in a second light emission state when the receiving unit receives input operation in a state that the light emitting unit is connected to the apparatus body.

### CROSS-REFERENCE TO RELATED APPLICATIONS

This application is based upon and claims the benefit of priority from Japanese Patent Application No. 2012-056120, filed on Mar. 13, 2012; and Japanese Patent Application No. 2013-029116, filed on Feb. 18, 2013, the entire contents of all of which are incorporated herein by reference.

### FIELD

Embodiments described herein relate generally to an ultrasonic probe and an ultrasound diagnostic apparatus.

### BACKGROUND

An ultrasound diagnostic apparatus radiates an ultrasonic pulse generated from an oscillating element installed on an ultrasonic probe into the body of a subject and receives an ultrasonic reflected wave caused by a difference between acoustic impedances of body tissues of the subject through the oscillating element, thereby collecting biological information. The ultrasound diagnostic apparatus is widely used for shape diagnostic and functional diagnostic of various organs, for example, because the ultrasound diagnostic apparatus enables real-time display of ultrasonic image data with simple operation such as touching with the ultrasonic probe.

Some ultrasound diagnostic apparatuses can be installed with a plurality of ultrasonic probes, which can be appropriately exchanged for use in accordance with diagnostic purposes such as the region to be tested and the conditions of the subject. The ultrasonic probes are detachably attached to the body of the ultrasound diagnostic apparatus through a connector. For example, three types of ultrasonic probes can be connected to the body of the ultrasound diagnostic apparatus through connectors and switch operation can be performed at the body side of the ultrasound diagnostic apparatus, enabling selective use of any of the ultrasonic probes. With the conventional technology, however, there have been some cases where diagnostic efficiency is lowered when a plurality of ultrasonic probes are switched over for use in diagnosis.

### BRIEF DESCRIPTION OF THE DRAWINGS

FIG. 1 is a diagram illustrating the overall structure of an ultrasound diagnostic apparatus according to a first embodiment;

FIG. 2 is a flowchart illustrating a conventional procedure of diagnosis using an ultrasound diagnostic apparatus;

FIG. 3 is a diagram illustrating an example of the structure of an ultrasonic probe according to the first embodiment;

FIG. 4 is a diagram illustrating light emission performed by a light emitting unit according to the first embodiment;

FIG. 5 is a diagram illustrating an example of the structure of a probe interface unit according to the first embodiment;

FIG. 6 is a flowchart illustrating the procedure of processing performed by the ultrasound diagnostic apparatus according to the first embodiment;

FIG. 7 is a flowchart illustrating the procedure of diagnosis using the ultrasound diagnostic apparatus according to the first embodiment;

FIG. 8 is a diagram illustrating light emission performed by a light emitting unit according to a second embodiment;

FIG. 9 is a flowchart illustrating the procedure of processing performed by an ultrasound diagnostic apparatus according to the second embodiment;

FIG. 10 is a diagram illustrating an example of the structure of an ultrasonic probe according to a third embodiment;

FIG. 11 is a diagram illustrating an example of the structure of a probe interface unit according to the third embodiment;

FIG. 12A is a diagram illustrating an example of the structure of the ultrasonic probe according to the third embodiment; and

FIG. 12B is a diagram illustrating another example of the structure of the ultrasonic probe according to the third embodiment.

### DETAILED DESCRIPTION

### First Embodiment

According to an embodiment, an ultrasonic probe having a piezoelectric vibrator transmitting and receiving ultrasonic waves under control of an apparatus body, the ultrasonic probe comprising a receiving unit and a light emitting unit. The receiving unit configured to receive input operation related to transmission and reception of ultrasonic waves performed by the piezoelectric vibrator. The light emitting unit configured to emit light in a first light emission state when connected to the apparatus body and emit light in a second light emission state when the receiving unit receives input operation in a state that the light emitting unit is connected to the apparatus body.

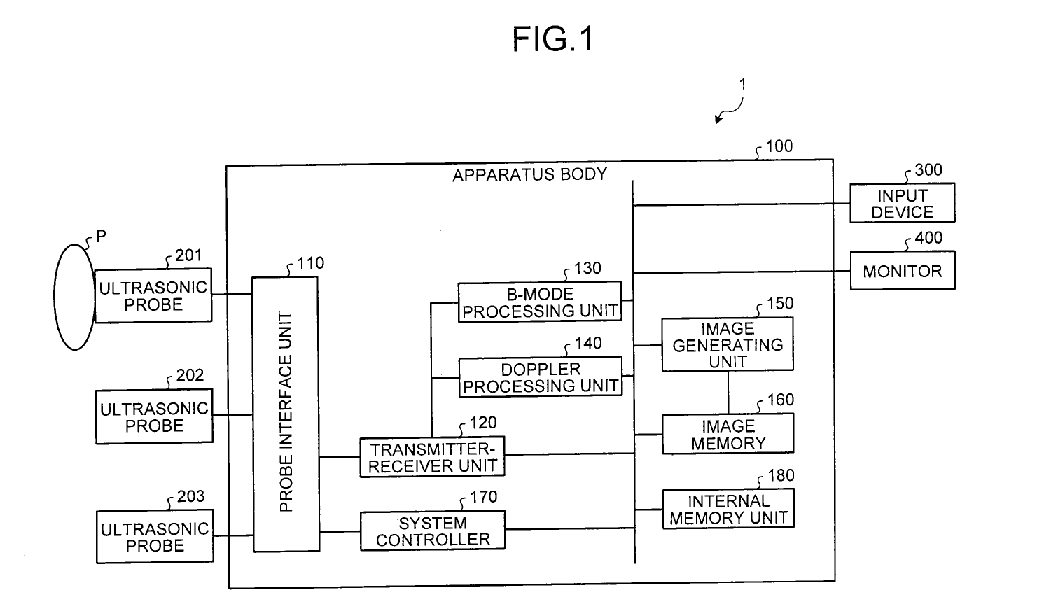

Firstly, the overall structure of an ultrasound diagnostic apparatus according to a first embodiment is described below with reference to FIG. 1. FIG. 1 is a diagram illustrating the overall structure of this ultrasound diagnostic apparatus 1 according to the first embodiment. As illustrated in FIG. 1, the ultrasound diagnostic apparatus 1 according to the first embodiment includes ultrasonic probes 201 to 203, an input device 300, a monitor 400, and an apparatus body 100.

The ultrasonic probes 201 to 203 include a plurality of piezoelectric vibrators. The piezoelectric vibrators generate ultrasonic waves based on drive signals supplied from a transmitter-receiver unit 120 included in the apparatus body 100 described later. The piezoelectric vibrators receive reflected waves from a subject P and convert the reflected waves thus received into electrical signals. Furthermore, the ultrasonic probes 201 to 203 include matching layers provided to the piezoelectric vibrators and backing materials preventing ultrasonic waves from traveling behind the piezoelectric vibrators, for example.

When ultrasonic waves are transmitted from the ultrasonic probe 201 to the subject P, for example, the ultrasonic waves thus transmitted are continuously reflected on the planes of discontinuity of the acoustic impedances in body tissues of the subject P and then received by the piezoelectric vibrators included in the ultrasonic probe 201 as reflected wave signals. The amplitudes of the reflected wave signals thus received depend on the differences between the acoustic impedances on the plane of discontinuity on which the ultrasonic waves are reflected. When the ultrasonic pulse transmitted is reflected on a moving blood flow or the surface of a cardiac wall, for example, the reflected wave signal undergoes a frequency shift depending on the velocity component in the ultrasound transmission direction of the moving body because of the Doppler effect. Similarly, the ultrasonic probes 202 and 203 also transmit ultrasonic waves and receive reflected waves.

Each of the ultrasonic probes 201 to 203 is of a different type such as a sector type, a linear type, or a convex type. In accordance with a diagnostic purpose, a suitable ultrasonic probe is selectively used. When used, an ultrasonic probe is changed from a non-active state, which is a waiting state, to an active state, in which ultrasonic waves can be transmitted and reflected waves can be received. The ultrasonic probes 201 to 203 according to the embodiment are configured to improve diagnostic efficiency in the switching described above. Details of the configuration are described below.

In the present embodiment, the ultrasonic probes may be one-dimensional ultrasonic probes with a plurality of piezoelectric vibrators installed in line. The ultrasonic probes may be ultrasonic probes in which the piezoelectric vibrators of the one-dimensional ultrasonic probe are mechanically vibrated, or two-dimensional ultrasonic probes with a plurality of piezoelectric vibrators installed two-dimensionally in a reticular pattern.

Although only three ultrasonic probes are illustrated in FIG. 1, the embodiment should not be limited thereto, and any number of ultrasonic probes may be provided. For example, four or more ultrasonic probes may be provided.

The input device 300 includes a trackball, a switch, buttons, and an operation panel. The input device 300 receives various setting requests from the operator of the ultrasound diagnostic apparatus 1 and transmits the setting requests thus received to the apparatus body 100.

The monitor 400 displays a graphical user interface (GUI) through which the operator of the ultrasound diagnostic apparatus 1 inputs various setting requests using the input device 300 and displays ultrasonic images generated by the apparatus body 100, for example.

The apparatus body 100 generates an ultrasonic image based on reflected waves received by the ultrasonic probes 201 to 203. As illustrated in FIG. 1, the apparatus body 100 includes a probe interface unit 110, the transmitter-receiver unit 120, a B-mode processing unit 130, a Doppler processing unit 140, an image generating unit 150, an image memory 160, a system controller 170, and an internal storage unit 180.

The probe interface unit 110 includes a connector to which each of the ultrasonic probes 201 to 203 is connected. The connector connects each of the ultrasonic probes 201 to 203 to the apparatus body 100. The probe interface unit 110 performs processing related to the switching of the ultrasonic probes 201 to 203. The processing related to the switching will be described later.

The transmitter-receiver unit 120 includes a trigger generation circuit, a delay circuit, and a pulsar circuit, and supplies drive signals to the ultrasonic probes 201 to 203. The pulsar circuit repeatedly generates a rate pulse for forming a transmission ultrasonic wave at a predefined rate frequency. The delay circuit provides each rate pulse generated by the pulsar circuit with a delay time for each piezoelectric vibrator. The delay time is required to converge ultrasonic waves generated by the ultrasonic probes 201 to 203 into a beam to determine transmission directionality. The trigger generation circuit applies a drive signal (drive pulse) to each of the ultrasonic probes 201 to 203 at the timing based on the rate pulse. In other words, the delay circuit adjusts the transmission direction from the surface of the piezoelectric vibrators as required by changing the delay time provided to each rate pulse.

The transmitter-receiver unit 120 includes an amplifier circuit, an analog/digital (A/D) converter, and an adder, and generates reflected wave data through various processing on reflected wave signals received by the ultrasonic probes 201 to 203. The amplifier circuit amplifies the reflected wave signal for each channel to perform gain correction processing. The A/D converter A/D-converts the reflected wave signal thus gain-corrected and provides a delay time required to determine reception directionality. The adder performs addition processing on the reflected wave signal processed by the A/D converter to generate reflected wave data. The addition processing performed by the adder enhances reflection components along the direction in accordance with the reception directionality of the reflected wave signal.

As described above, the transmitter-receiver unit 120 controls transmission and reception directionalities in the transmission and reception of the ultrasonic wave, respectively. The transmitter-receiver unit 120 has a function of instantaneously changing delay information, transmission frequency, transmission drive voltage, the number of aperture elements, and the like under the control of the system controller 170 described later. In particular, the transmission drive voltage is changed by a linear amplifier oscillation circuit capable of instantaneously changing values or a mechanism that electrically switches a plurality of power source units. The transmitter-receiver unit 120 is also capable of transmitting and receiving a different waveform for each frame or rate.

The B-mode processing unit 130 receives reflected wave data, which is a processed reflected wave signal that has gone through the gain correction processing, A/D conversion processing, and the addition processing, from the transmitter-receiver unit 120. The B-mode processing unit 130 then performs logarithmic amplification, envelope demodulation, and the like to generate data in which the intensity of a signal is represented by the brightness of its luminance (B-mode data).

The Doppler processing unit 140 performs frequency analysis of velocity information from the reflected wave data received from the transmitter-receiver unit 120 and extracts blood flow component, tissue component, and contrast agent echo component that are affected by the Doppler effect, thereby generating data extracted at multiple points from moving body information, such as average velocity, variance, and power (Doppler data).

The image generating unit 150 generates an ultrasonic image from the B-mode data generated by the B-mode processing unit 130 and the Doppler data generated by the Doppler processing unit 140. Specifically, the image generating unit 150 generates an ultrasonic image for display (B-mode image and Doppler image) from the B-mode data and the Doppler data through conversion (scan-conversion) of a scan line signal array resulting from ultrasonic scanning into a scan line signal array in a video format represented by television.

The image memory 160 stores therein image data such as a contrast image and a tissue image generated by the image generating unit 150. The image memory 160 also stores therein results of the processing performed by the system controller 170 described later. The image memory 160 also stores therein an output signal (radio frequency (RF)) and a luminance signal of an image that have just passed through the transmitter-receiver unit 120, various raw data, image data acquired via a network, and the like as necessary. The data format of the image data stored in the image memory 160 may be a data format after video format conversion and displayed on the monitor 400 under the control of the system controller 170 described later, or may be a data format before coordinate conversion that is raw data generated by the B-mode processing unit 130 and the Doppler processing unit 140.

The system controller 170 controls the overall processing performed by the ultrasound diagnostic apparatus 1. Specifically, the system controller 170 controls processing performed by the probe interface unit 110, the transmitter-receiver unit 120, the B-mode processing unit 130, the Doppler processing unit 140, and the image generating unit 150 based on various setting requests received from the operator through the input device 300 and various control programs and setting information read from the internal storage unit 180. The system controller 170 also controls the monitor 400 to display an ultrasonic image stored in the image memory 160. For example, the system controller 170 calculates control data for a case of using an ultrasonic probe switched to the active state based on characteristics information for each probe stored in the internal storage unit 180 described later. Thereafter, the system controller 170 sets the control data thus calculated to the probe interface unit 110, the transmitter-receiver unit 120, the B-mode processing unit 130, the Doppler processing unit 140, and the image generating unit 150.

The internal storage unit 180 stores therein various data such as control programs for performing transmission and reception of ultrasonic waves, image processing, and display processing; diagnostic information (subjects' IDs and doctors' opinions, for example); and a diagnostic protocol. The internal storage unit 180 is also used for storing therein images stored in the image memory 160 as necessary. Furthermore, the internal storage unit 180 stores therein the characteristics information for each ultrasonic probe used by the system controller 170. For example, the internal storage unit 180 stores therein, for example, characteristics information on an identifier for identifying an ultrasonic probe (a model name, for example) associated with transmission and reception characteristics of ultrasonic waves.

The overall structure of the ultrasound diagnostic apparatus according to the first embodiment has been described above. Based on such a structure, the ultrasound diagnostic apparatus 1 according to the first embodiment is configured to improve diagnostic efficiency when a plurality of ultrasonic probes are switched for use in diagnosis through the operation of the ultrasonic probes 201 to 203 and the probe interface unit 110, which will be described in detail later.

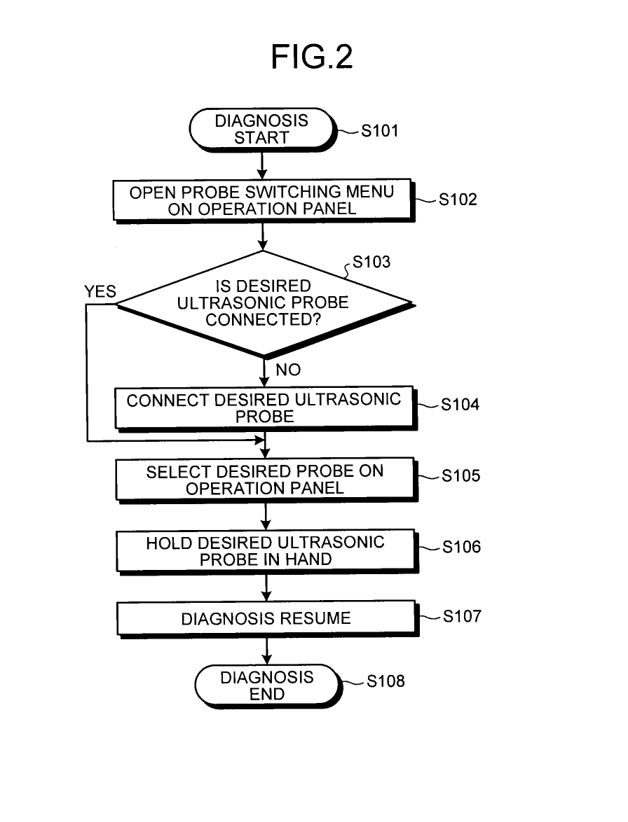

Conventional switching of ultrasonic probes will be described here. FIG. 2 is a flowchart illustrating a conventional procedure of diagnosis using an ultrasound diagnostic apparatus. Conventionally, when an operator starts diagnosis (Step S101), the operator operates an operation panel to open a probe switching menu (Step S102) and checks if the desired ultrasonic probe is connected (Step S103) as illustrated in FIG. 2. The probe switching menu is a screen for switching ultrasonic probes displayed on a predefined display unit (a touch command screen included in the operation panel, for example).

Specifically, the operator checks if the desired ultrasonic probe is included in a list of probes displayed on the probe switching menu. When the desired ultrasonic probe is not connected (not included in the list of probes) (No at Step S103), the operator connects the desired ultrasonic probe to the probe interface unit (Step S104) and selects the desired ultrasonic probe on the operation panel (Step S105).

When the desired ultrasonic probe is connected (included in the list of probes) (Yes at Step S103), the operator selects the desired probe on the operation panel (Step S105). The list of probes is information indicating a list of ultrasonic probes connected to the probe interface unit 110.

Thereafter, the operator holds the desired ultrasonic probe in hand (Step S106) and resumes the diagnosis (Step S107). Then, the diagnosis is completed (Step S108). As described above, to switch ultrasonic probes with the conventional technology, the operator operates the operation panel to display the probe switching menu and check connection of an ultrasonic probe to select the ultrasonic probe.

Thus, each time a plurality of ultrasonic probes are switched over for use in diagnosis with the conventional technology, the operator has to operate the operation panel, lowering the diagnostic efficiency. To solve this problem, the ultrasound diagnostic apparatus 1 according to the first embodiment is configured to improve diagnostic efficiency when a plurality of ultrasonic probes are switched over for use in diagnosis.

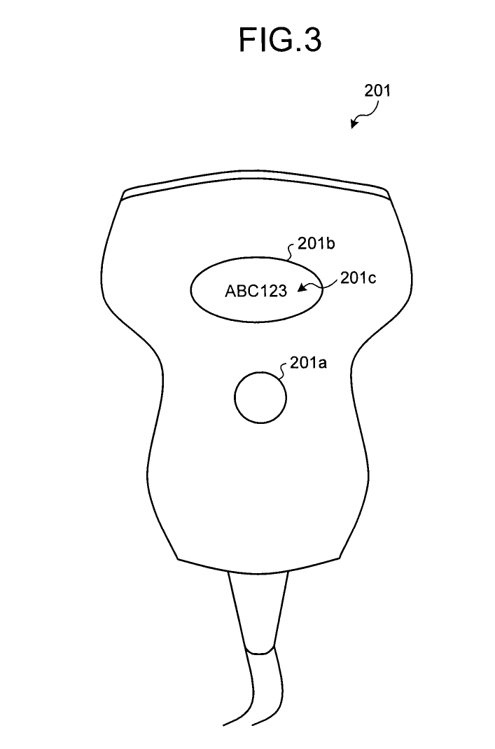

The ultrasonic probes 201 to 203 and the probe interface unit 110 according to the first embodiment are described below with reference to FIG. 3. FIG. 3 is a diagram illustrating an example of the structure of the ultrasonic probe 201 according to the first embodiment. Although FIG. 3 exemplifies the ultrasonic probe 201, the ultrasonic probes 202 and 203 have the same structure.

As illustrated in FIG. 3, the ultrasonic probe 201 according to the first embodiment includes a press-down button 201a, a light emitting unit 201b, and a model name 201c. The press-down button 201a receives input operation related to transmission and reception of ultrasonic waves performed by the piezoelectric vibrators. For example, the press-down button 201a receives press-down operation to switch the ultrasonic probe 201 to the active state when the operator performs diagnosis using the ultrasonic probe 201.

To cite an example, the press-down button 201a is pressed down by the operator with the ultrasonic probe 201 connected to the probe interface unit 110. The press-down button 201a then transmits a press-down signal indicating that the button is being pressed down to the probe interface unit 110.

The light emitting unit 201b emits light in a first light emission state when connected to the apparatus body 100, and emits light in a second light emission state when the input operation is received by the press-down button 201a, while being connected to the apparatus body 100. Specifically, the light emitting unit 201b emits light in a state that at least one of the color of the light, lighting, blinking, the intensity of the light is changed between the first light emission state and the second light emission state.

For example, the light emitting unit 201b emits light in a predefined color when connected to the apparatus body 100 and emits light in a color different from the predefined color when the input operation is received by the press-down button 201a. To cite an example, the light emitting unit 201b emits light in a predefined color based on a first light emission signal received from the probe interface unit 110 when the ultrasonic probe 201 is connected to the probe interface unit 110. The light emitting unit 201b emits light in a color different from the predefined color based on a second light emission signal transmitted from the probe interface unit 110 when the press-down button 201a is pressed down.

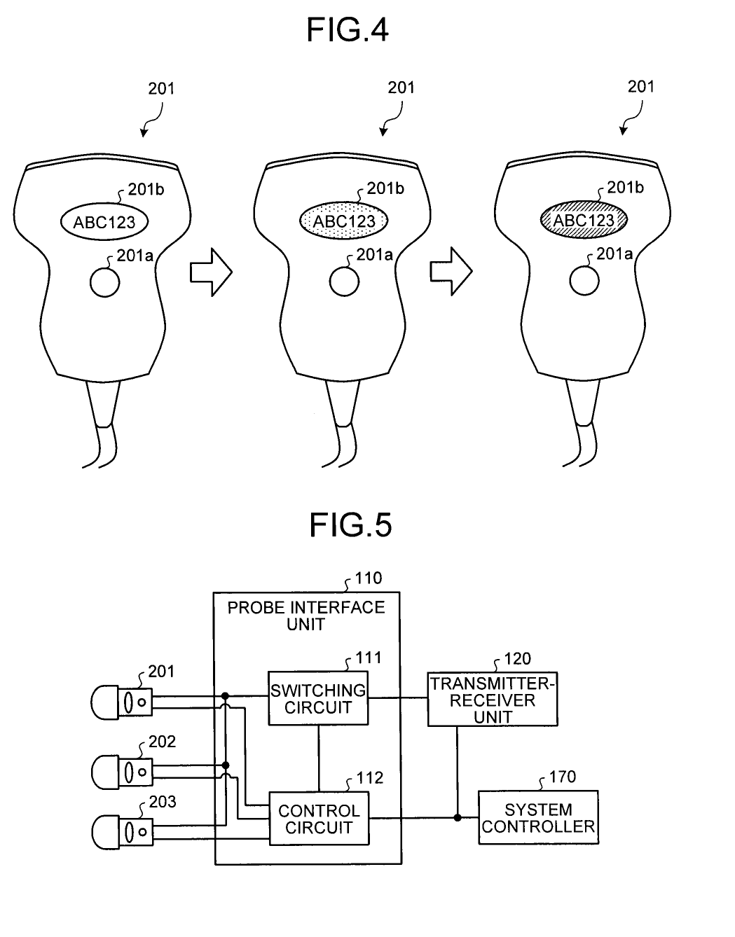

FIG. 4 is a diagram illustrating light emission performed by the light emitting unit 201b according to the first embodiment. For example, the light emitting unit 201b is in a turned-off state, as illustrated in the left figure of FIG. 4, when the ultrasonic probe 201 is not connected to the probe interface unit 110. When the ultrasonic probe 201 is connected to the probe interface unit 110, the probe interface unit 110 is notified of the connection through a connector (not illustrated) of the ultrasonic probe.

When notified of the connection through the connector of the ultrasonic probe 201, the probe interface unit 110 transmits the first light emission signal to the ultrasonic probe 201 for causing the light emitting unit 201b to emit light. Upon receiving the first light emission signal from the probe interface unit 110, the light emitting unit 201b emits light in a predefined color based on the first light emission signal thus received as illustrated in the middle diagram in FIG. 4.

Thereafter, when the press-down button 201a is pressed down by the operator, a press-down signal is transmitted from the press-down button 201a to the probe interface unit 110. Upon receiving the press-down signal, the probe interface unit 110 transmits the second light emission signal to the ultrasonic probe 201. Upon receiving the second light emission signal from the probe interface unit 110, the light emitting unit 201b emits light in a color different from the predefined color (the color of the light emitting unit 201b in the middle figure in FIG. 4) based on the second light emission signal thus received as illustrated in the right figure in FIG. 4.

The light emitting unit 201b illustrated in FIG. 3 has a front face made of a transparent plastic material, behind which a light emitting diode (LED) or the like is embedded in the ultrasonic probe 201. The colors generated by the first light emission signal and the second light emission signal can be set to any colors. The light emitted by the light emitting unit 201b may be emitted by a single LED or a plurality of LEDs. Specifically, the color of the light emitted by the light emitting unit 201b may be changed by a single LED or changed by a plurality of LEDs generating different colors through lighting in accordance with the states.

The example above describes a case where the color changes between the first light emission state and the second light emission state. The embodiment is, however, not limited thereto, but is also applicable to a case where a lighting state changes to a blinking state or a blinking state changes to a lighting state, instead of the color change between the first light emission state and the second light emission state. Also applicable is a case where the intensity of the light changes between the first light emission state and the second light emission state instead of the color change. Also, between the first light emission state and the second light emission state, the color of the light, lighting, blinking, or the intensity of the light may change in any combination, for example.

The light emitting unit is installed in a position at the piezoelectric vibrator side relative to the press-down button. For example, as illustrated in FIG. 3, the light emitting unit 201b is disposed at the piezoelectric vibrator side relative to the press-down button 201a on the ultrasonic probe 201, thus preventing the light emitting unit being covered by the operator's hand or fingers when the operator presses down the press-down button.

Referring back to FIG. 3, the ultrasonic probe 201 is provided with identification information for identifying the ultrasonic probe. For example, the ultrasonic probe 201 includes the model name 201c as illustrated in FIG. 3. The identification information (the model name 201c, for example) is irradiated with light emitted by the light emitting unit 201b. To cite an example, the model name 201c indicates the type of the ultrasonic probe 201 as illustrated in FIG. 3 and is disposed on the front face of or near the light emitting unit 201b. For example, the model name 201c indicates if the ultrasonic probe 201 is of a sector type, a linear type, or a convex type. The model name 201c also indicates characteristics such as the median frequency of the ultrasonic probe 201, for example. The model name 201c also indicates if the ultrasonic probe 201 is of a two-dimensional scanning type or a three-dimensional scanning type, for example. The model name 201c is printed on the plastic portion on the front face of the light emitting unit 201b, for example, as illustrated in FIG. 3.

Next, the probe interface unit 110 will be described. FIG. 5 is a diagram illustrating an example of the structure of the probe interface unit 110 according to the first embodiment. As illustrated in FIG. 5, the probe interface unit 110 includes a switching circuit 111 and a control circuit 112 and is connected to the ultrasonic probes 201 to 203.

The switching circuit 111 switches ultrasonic probes to be controlled by the transmitter-receiver unit 120 controlling the piezoelectric vibrators so as to transmit and receive ultrasonic waves. Specifically, the switching circuit 111 switches, under the control of the control circuit 112, the destination of a control signal for controlling the piezoelectric vibrators transmitted by the transmitter-receiver unit 120 to an ultrasonic probe for which the press-down button is pressed down. For example, the switching circuit 111 switches the destination of the control signal transmitted by the transmitter-receiver unit 120 to the ultrasonic probe 201.

The control circuit 112 controls the piezoelectric vibrators of the ultrasonic probe to be controlled by the transmitter-receiver unit 120 when the press-down button included in the ultrasonic probe is pressed down while the ultrasonic probe is connected to the probe interface unit 110. Specifically, the control circuit 112 receives a press-down signal from the ultrasonic probe and controls the switching circuit 111 so that a control signal from the transmitter-receiver unit 120 is transmitted to the ultrasonic probe from which the press-down signal thus received has been transmitted.

For example, upon receiving the press-down signal from the press-down button 201a of the ultrasonic probe 201, the control circuit 112 controls the switching circuit 111 to set the destination of the control signal transmitted by the transmitter-receiver unit 120 to the ultrasonic probe 201. Thereafter, the control circuit 112 transmits the identification information (model name, for example) of the ultrasonic probe 201 from which the press-down signal has been transmitted, to the system controller 170. Thus, the system controller 170 reads out characteristic information corresponding to the identification information thus received, calculates control data, and configures the transmitter-receiver unit 120, the B-mode processing unit 130, the Doppler processing unit 140, the image generating unit 150, and the like.

When the ultrasonic probe 201 is connected to the probe interface unit 110, the control circuit 112 controls the light emitting unit 201b included in the ultrasonic probe 201 to emit light in the first light emission state. When input operation is received by the press-down button 201a while the ultrasonic probe 201 is connected to the probe interface unit 110, the control circuit 112 controls the light emitting unit 201b to emit light in the second light emission state. For example, the control circuit 112 controls the light emitting unit 201b included in the ultrasonic probe 201 to emit light in a predefined color when the ultrasonic probe 201 is connected to the probe interface unit 110. To cite an example, when notified of the connection through the connector of the ultrasonic probe 201, the control circuit 112 controls the light emitting unit 201b of the ultrasonic probe to emit light in a predefined color by transmitting the first light emission signal to the light emitting unit 201b. For example, when notified of the connection through the connector of the ultrasonic probe 201, the control circuit 112 transmits the first light emission signal to the light emitting unit 201b.

Thereafter, the control circuit 112 controls the light emitting unit to emit light in a color different from the predefined color when the press-down button is pressed down while the ultrasonic probe is connected to the probe interface unit 110. Specifically, upon receiving a pressed-down signal, the control circuit 112 transmits the second light emission signal, to the light emitting unit of the ultrasonic probe from which the pressed-down signal has been transmitted, for controlling the light emitting unit to emit light in a different color from that caused by the first light emission signal. For example, upon receiving the press-down signal from the press-down button 201a with the ultrasonic probe 201 connected to the probe interface unit 110, the control circuit 112 transmits the second light emission signal to the light emitting unit 201b. The switching control of the light emission signal described above may be controlled directly by the control circuit 112 or by the control circuit 112 under the control of the system controller 170. The example above describes a case where the control circuit 112 changes the color of the light emitting unit between the first emission state and the second emission state. The control circuit 112 can also control the light emission state when controlling lighting, blinking, and the intensity of the light in the same way as in the above-described example. Specifically, the control circuit 112 transmits the light emission signal, to the light emitting unit, for controlling the light emitting unit to emit light in each light emission state.

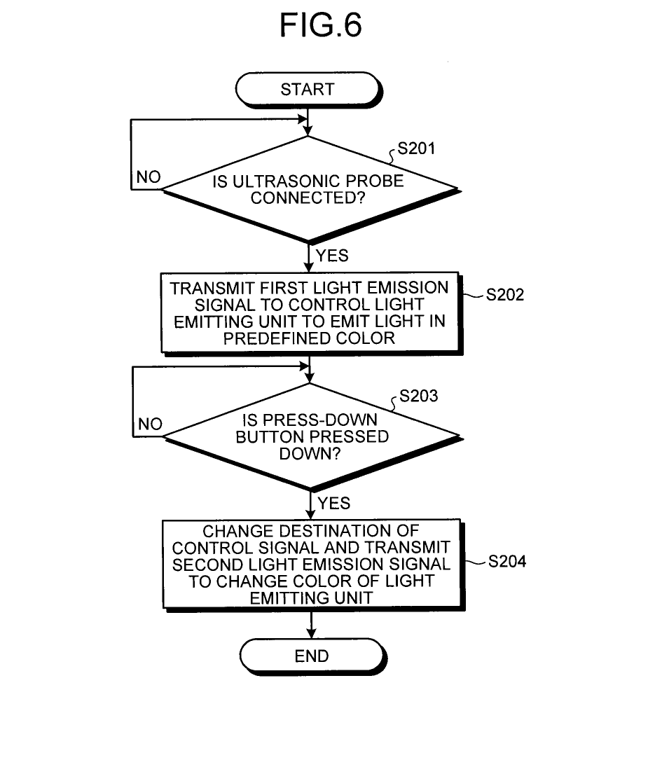

Next, described will be the procedure of processing performed by the ultrasound diagnostic apparatus 1 according to the first embodiment. FIG. 6 is a flowchart illustrating the procedure of processing performed by the ultrasound diagnostic apparatus 1 according to the first embodiment. FIG. 6 illustrates a case where the colors of the light are changed between the first light emission state and the second light emission state. In the ultrasound diagnostic apparatus 1 according to the first embodiment, when the ultrasonic probe is connected to the probe interface unit 110 (Yes at Step S201), the control circuit 112 transmits the first light emission signal to the ultrasonic probe that has been notified of the connection, and controls the light emitting unit of the ultrasonic probe to emit light in a predefined color (Step S202) as illustrated in FIG. 6.

Thereafter, when the press-down button of the ultrasonic probe connected to the probe interface unit 110 is pressed down (Yes at Step S203), the control circuit 112 receives the press-down signal and controls the switching circuit to change the destination of the control signal transmitted by the transmitter-receiver unit 120 to the ultrasonic probe from which the press-down signal has been transmitted, while transmitting the second light emission signal to the ultrasonic probe to change the color of the light emitting unit (Step S204).

Then, the ultrasound diagnostic apparatus 1 according to the first embodiment ends the procedures following a diagnosis ending operation performed by the operator. The ultrasound diagnostic apparatus 1 according to the first embodiment is in a waiting state until the ultrasonic probe is connected (No at Step S201). The ultrasound diagnostic apparatus 1 according to the first embodiment is also in a waiting state until the press-down button is pressed down (No at Step S203).

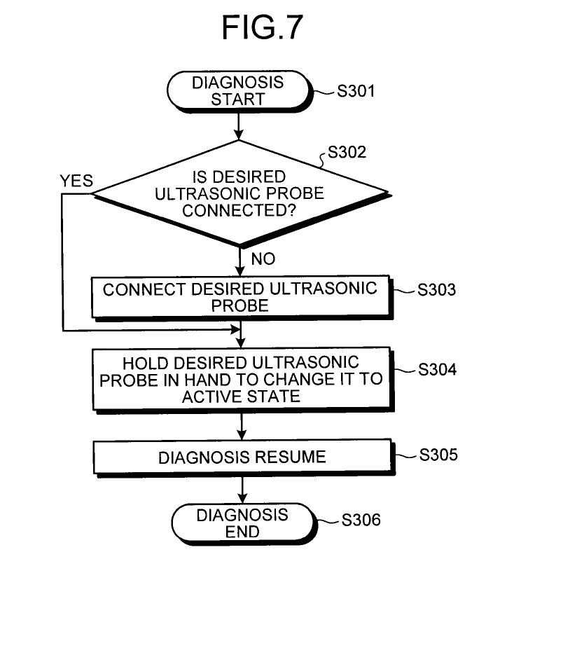

Next, described will be the procedure of switching of ultrasonic probes performed by the ultrasound diagnostic apparatus 1 according to the first embodiment. FIG. 7 is a flowchart illustrating the procedure of diagnosis using the ultrasound diagnostic apparatus 1 according to the first embodiment. In the ultrasound diagnostic apparatus 1 according to the first embodiment, when the operator starts diagnosis (Step S301), the operator checks if the desired ultrasonic probe is connected (Step S302) as illustrated in FIG. 7.

Specifically, the operator checks if the light emitting unit of the desired ultrasonic probe is turned on. When the desired ultrasonic probe is not connected (the light emitting unit is turned off) (No at Step S302), the operator connects the desired ultrasonic probe to the probe interface unit 110 (Step S303).

Thereafter, the operator holds the desired ultrasonic probe in hand and changes the state thereof to the active state (Step S304) and resumes the diagnosis (Step S305). Specifically, the operator presses down the press-down button of the desired ultrasonic probe to resume the diagnosis.

When the desired ultrasonic probe is connected (the light emitting unit is turned on) (Yes at Step S302), the operator holds the desired ultrasonic probe in hand and changes the state thereof to the active state (Step S304), resuming the diagnosis (Step S305). Then, the diagnosis is completed (Step S306).

As described above, the press-down button 201a receives input operation for starting transmission and reception of ultrasonic waves performed by the piezoelectric vibrators according to the first embodiment. The ultrasonic probe 201 according to the first embodiment can thus switch over ultrasonic probes without operation on the operation panel, thereby improving diagnostic efficiency when a plurality of ultrasonic probes are switched over for use in diagnosis.

According to the first embodiment, the light emitting unit emits light in the first light emission state when the ultrasonic probe is connected to the probe interface unit 110, and emits light in the second light emission state when the input operation is received through the press-down button while the ultrasonic probe is connected to the probe interface unit 110. With The ultrasonic probe 201 according to the first embodiment, the state of an ultrasonic probe can thus be checked by simply seeing the ultrasonic probe, thereby improving diagnostic efficiency when a plurality of ultrasonic probes are switched over for use in diagnosis.

According to the first embodiment, the light emitting unit emits light in a state that at least one of the color of the light, lighting, blinking, and the intensity of the light is changed between the first light emission state and the second light emission state. For example, the light emitting unit 201b emits light in a predefined color when the light emitting unit 201b is connected to the apparatus body and emits light in a color different from the predefined color when input operation is received by the press-down button 201a. With the ultrasonic probe 201 according to the first embodiment, the connection state of the ultrasonic probe to the probe interface unit 110 and the active state, for example, can thus be checked in one glance without operation on the operation panel, thereby improving diagnostic efficiency when a plurality of ultrasonic probes are switched over for use in diagnosis.

According to the first embodiment, the light emitting unit 201b is installed in a position on the ultrasonic probe 201 at the piezoelectric vibrator side relative to the press-down button 201a. The ultrasonic probe 201 according to the first embodiment can thus prevent the light emitting unit 201b from being covered by the operator's hand or fingers when the operator presses down the press-down button, allowing the operator to constantly perform visual check of the light emitting unit 201b.

According to the first embodiment, the model name 201c indicates the type of the ultrasonic probe and is disposed on the front face of or near the light emitting unit 201b. With the ultrasonic probe 201 according to the first embodiment, the operator can thus identify the ultrasonic probe in one glance while checking the connection state of the ultrasonic probe and the active state, for example, thereby further improving diagnostic efficiency when a plurality of ultrasonic probes are switched over for use in diagnosis.

According to the first embodiment, the control circuit 112 controls the switching circuit 111, when the press-down button included in the ultrasonic probe is pressed down while the ultrasonic probe is connected to the probe interface unit 110, so that the transmitter-receiver unit 120 controls the piezoelectric vibrators of the ultrasonic probe. The ultrasound diagnostic apparatus 1 according to the first embodiment thus improves diagnostic efficiency when a plurality of ultrasonic probes are switched over for use in diagnosis.

According to the first embodiment, the control circuit 112 controls the light emitting unit to emit light in the first light emission state when the ultrasonic probe is connected to the probe interface unit 110, and to emit light in the second light emission state when input operation is received by the press-down button while the ultrasonic probe is connected to the probe interface unit 110. With the ultrasound diagnostic apparatus 1 according to the first embodiment, the state of the ultrasonic probe can thus be checked by simply seeing the ultrasonic probe, improving diagnostic efficiency when a plurality of ultrasonic probes are switched over for use in diagnosis.

For example, the control circuit 112 controls the light emitting element included in the ultrasonic probe to emit light in a predefined color when the ultrasonic probe is connected to the probe interface unit 110, and controls the light emitting element to emit light in a color different from the predefined color when the press-down button is pressed down while the ultrasonic probe is connected to the probe interface unit 110. With the ultrasound diagnostic apparatus 1 according to the first embodiment, the operator can thus check the state of the ultrasonic probe in one glance, thereby further improving diagnostic efficiency when a plurality of ultrasonic probes are switched over for use in diagnostic.

### Second Embodiment

The first embodiment described above has described a case where the light emitting unit emits light in a first light emission state when the ultrasonic probe is connected to the probe interface unit 110, and emits light in a second light emission state when the press-down button is pressed down while the ultrasonic probe is connected to the probe interface unit 110. A second embodiment will describe a case where the press-down button is pressed down again while the light emitting unit emits light in the second light emission state.

Although the ultrasonic probe 201 is described as an example in the following, the ultrasonic probes 202 and 203 have the same structure. For example, when the operator performs diagnosis using the ultrasonic probe 201, the press-down button 201a receives press-down operation for changing the ultrasonic probe 201 from an active state to a freeze state.

To cite an example, the press-down button 201a is pressed down by the operator while the ultrasonic probe 201 is in the active state. The press-down button 201a then transmits, to the probe interface unit 110, a press-down signal a signal indicating that the button is pressed down.

The light emitting unit 201b emits light in the light emission state as the second light emission state changed each time the press-down button 201a receives the input operation. For example, the light emitting unit 201b emits light in a color different from the color indicating the active state. To cite an example, when the ultrasonic probe 201 is connected to the probe interface unit 110, the light emitting unit 201b emits light in a predetermined color based on the first light emission signal received from the probe interface unit 110. The light emitting unit 201b emits light in a color different from the predefined color based on the second light emission signal transmitted from the probe interface unit 110 when the press-down button 201a is pressed down. The light emitting unit 201b emits light in another different color based on the third light emission signal transmitted from the probe interface unit 110 when the press-down button 201a is pressed down.

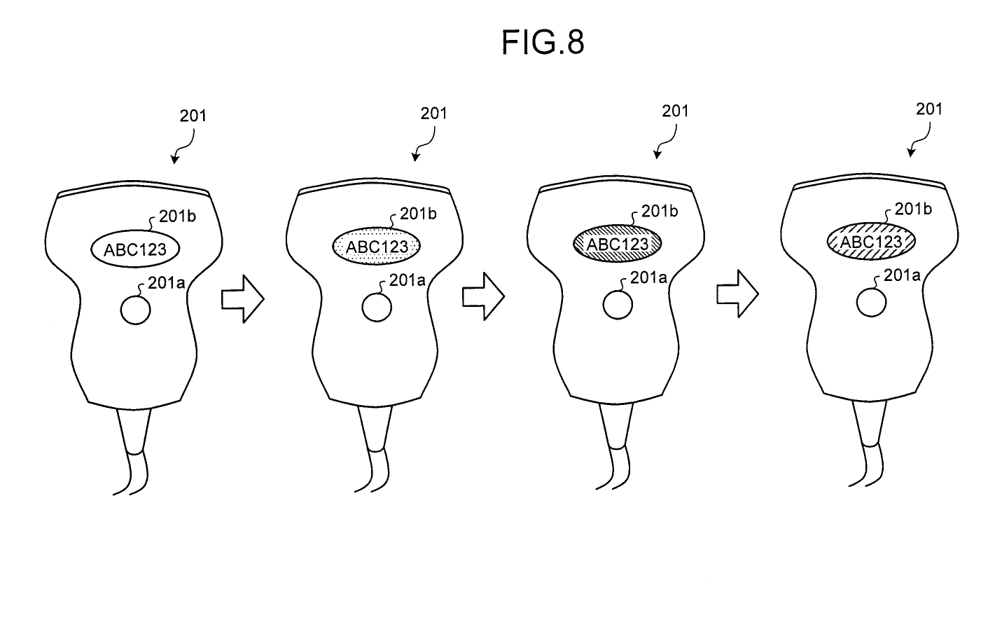

FIG. 8 is a diagram illustrating light emission performed by the light emitting unit 201b according to the second embodiment. The state of the ultrasonic probe 201 illustrated on the left in FIG. 8 is the same as in FIG. 4. Specifically, when the ultrasonic probe 201 is not connected to the probe interface unit 110, the light emitting unit 201b is in a turned-off state, as illustrated in the leftmost figure. When the ultrasonic probe 201 is connected to the probe interface unit 110, the light emitting unit 201b receives the first light emission signal from the probe interface unit 110 and emits light in a predefined color based on the first light emission signal thus received, as illustrated in the second leftmost figure in FIG. 8.

Thereafter, when the press-down button 201a is pressed down by the operator, the light emitting unit 201b receives the second light emission signal from the probe interface unit 110 and emits light in a different color from the predefined color (the color of the light emitting unit 201b in the second leftmost figure in FIG. 8) based on the second light emission signal thus received, as illustrated in the second rightmost figure in FIG. 8.

Furthermore, when the press-down button 201a is pressed down again by the operator, a press-down signal is transmitted from the press-down button 201a to the probe interface unit 110. Upon receiving the press-down signal, the probe interface unit 110 transmits the third light emission signal to the ultrasonic probe 201. Upon receiving the third light emission signal from the probe interface unit 110, the light emitting unit 201b emits light in a color different from the predefined color (the color of the light emitting unit 201b in the second rightmost figure in FIG. 8) based on the third light emission signal thus received, as illustrated in the rightmost figure in FIG. 8.

Although the example above describes a case where the color emitted by the light emitting unit 201b changes, the embodiment is not limited thereto. For example, lighting, blinking, or the intensity of the light may change. The example above also describes a case where, when the press-down button is pressed down while the ultrasonic probe 201 is in the active state, the state changes to the freeze state. The embodiment is, however, not limited thereto, but the mode, for example, may change. Specifically, the designer or the operator may set any function to be performed when the press-down button 201a is pressed down while the ultrasonic probe 201 is in the active state.

When the press-down button 201a is pressed down again while the ultrasonic probe 201 is in the freeze state, the freeze state may be lifted, or another function may be performed. For example, when the freeze state is lifted, the light emitting unit 201b may switch back in the light emission state in the active state (back in the state of the second rightmost figure in FIG. 8, for example). For example, when another function is performed, the light emitting unit 201b changes to another different light emission state. To cite an example, the light emitting unit 201b emits light in another different color (emit light in a color different from the color of the light emitting unit 201b in the rightmost figure in FIG. 8, for example). The control described above is controlled by light emitting signals received from the control circuit 112 described above.

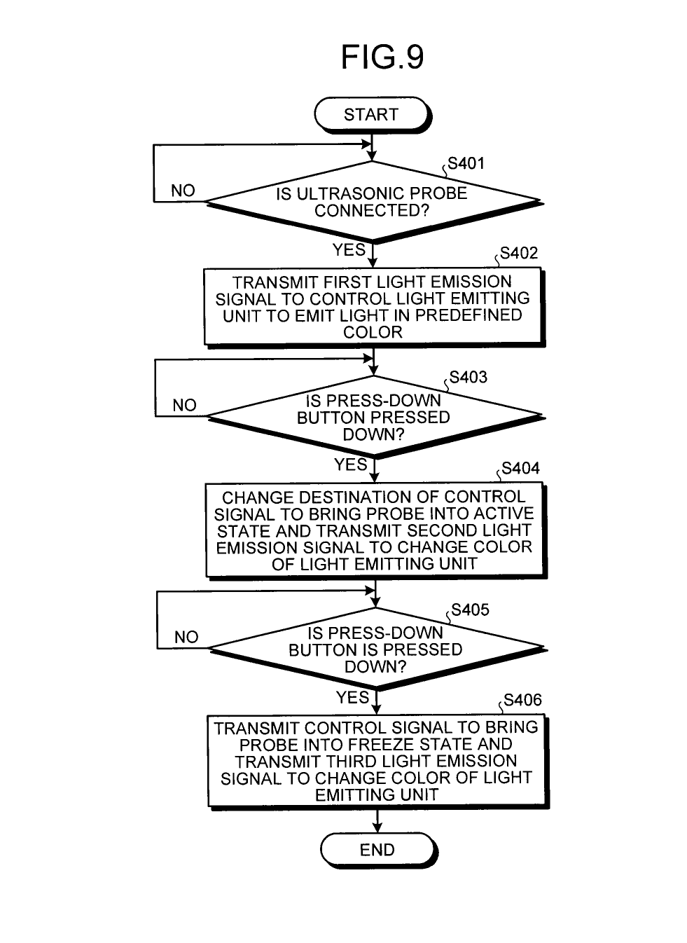

Next, described will be the procedure of the processing performed by the ultrasound diagnostic apparatus 1 according to the second embodiment. FIG. 9 is a flowchart illustrating the procedures of the processing performed by the ultrasound diagnostic apparatus according to the second embodiment. FIG. 9 illustrates a case where the color of the light changes between the first light emission state and the second light emission state. In the ultrasound diagnostic apparatus 1 according to the second embodiment, when the ultrasonic probe is connected to the probe interface unit 110 (Yes at Step S401), the control circuit 112 transmits the first light emission signal to the ultrasonic probe notified of the connection and controls the light emitting unit of the ultrasonic probe to emit light in a predefined color (Step S402) as illustrated in FIG. 9.

Thereafter, when the press-down button of the ultrasonic probe connected to the probe interface unit 110 is pressed down (Yes at Step S403), the control circuit 112 receives the press-down signal and controls the switching circuit to change the destination of the control signal transmitted by the transmitter-receiver unit 120 to the ultrasonic probe that has transmitted the press-down signal and switches the ultrasonic probe to the active state, while transmitting the second light emission signal to the ultrasonic probe to change the color of the light emitting unit (Step S404).

When the press-down button of the ultrasonic probe in the active state is pressed down (Yes at Step S405), the control circuit 112 receives the press-down signal and transmits a control signal to switch the ultrasonic probe into the freeze state while transmitting the third light emission signal to further change the color of the light emitting unit (Step S406).

Then, the ultrasound diagnostic apparatus 1 according to the second embodiment ends the processes following a diagnosis ending operation performed by the operator. The ultrasound diagnostic apparatus 1 according to the second embodiment is in a waiting state until the ultrasonic probe is connected (No at Step S401). The ultrasound diagnostic apparatus 1 according to the second embodiment is also in a waiting state until the press-down button is pressed down (No at Step S403). The ultrasound diagnostic apparatus 1 according to the second embodiment maintains the ultrasonic probe in the active state until the press-down button is pressed down (No at Step S405).

As described above, according to the second embodiment, the light emitting unit 201b emits light in the light emission state as the second light emission state changed each time the press-down button 201a receives input operation while the light emitting unit 201b is in the second light emission state. With the ultrasonic probe 201 according to the second embodiment, current statuses of various functions can thus be checked in one glance.

### Third Embodiment

While the first and second embodiments have been described above, various forms other than the first and second embodiments described above may be applicable.

The first and second embodiments described above have described a case where the ultrasonic probe and the apparatus body 100 are connected by a cable. The embodiments are, however, not limited thereto, but the ultrasonic probe and the apparatus body 100 may be connected through radio communication, for example. In such a case, for example, the ultrasonic probe and the apparatus body 100 each include a function part performing radio communication through which control signals related to the connection between the ultrasonic probe and the apparatus body 100, control signals related to the transmission and reception of ultrasonic waves, and the like are communicated. The following description will mainly describe such a case.

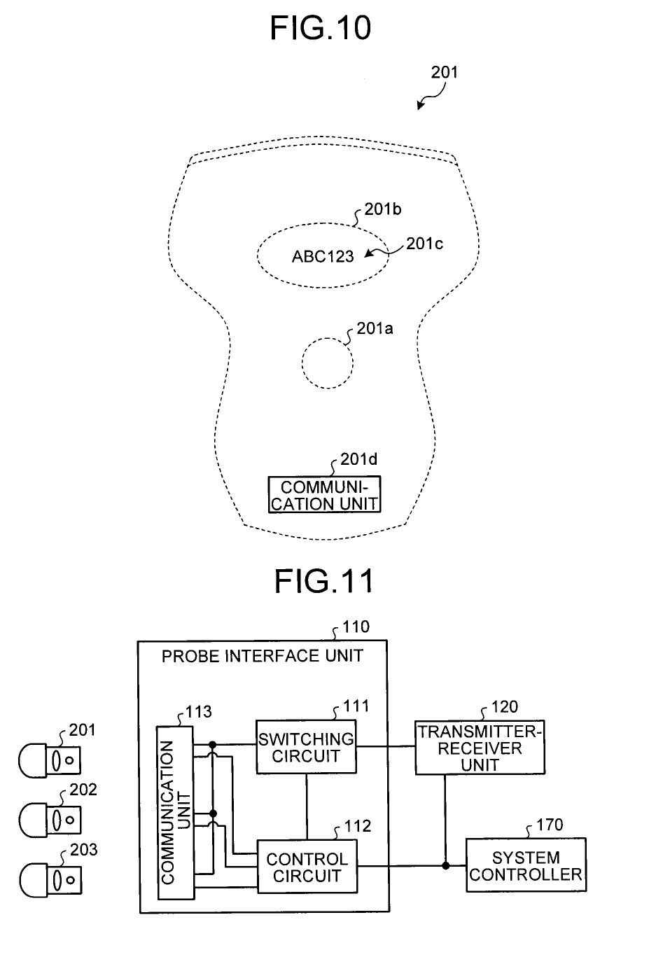

FIG. 10 is a diagram illustrating an example of the structure of an ultrasonic probe 201 according to a third embodiment. FIG. 10 exemplifies the ultrasonic probe 201. For example, the ultrasonic probe 201 according to the third embodiment includes a communication unit 201d in addition to the press-down button 201a, the light emitting unit 201b, and the model name 201c as illustrated in FIG. 10.

The communication unit 201d performs transmission and reception of a control signal with the apparatus body 100 through radio signals. Specifically, the communication unit 201d performs transmission and reception of, for example, a control signal related to the connection with the apparatus body 100 and a control signal related to transmission and reception of ultrasonic waves with a communication unit included in the apparatus body 100.

For example, the communication unit 201d determines whether the communication unit 201d is located within the range of communication with the apparatus body 100 by transmitting communication signals periodically. The communication unit 201d notifies the communication unit included in the apparatus body 100 of the connection through radio communication, when located in the range of communication with the apparatus body 100. When the press-down button 201a is pressed down while the connection through radio communication is established, the communication unit 201d transmits a press-down signal generated by the press-down button 201a to the apparatus body 100.

The communication unit 201d receives a control signal for controlling piezoelectric vibrators and a control signal for controlling the light emitting unit 201b to emit light from the communication unit of the apparatus body 100.

FIG. 11 is a diagram illustrating an example of the structure of a probe interface unit 110 according to the third embodiment. As illustrated in FIG. 11, the probe interface unit 110 according to the third embodiment includes a communication unit 113 in addition to the switching circuit 111 and the control circuit 112.

The communication unit 113 performs transmission and reception of a control signal with the ultrasonic probe through radio signals. Specifically, the communication unit 113 transmits and receives, for example, a control signal related to the connection with the ultrasonic probe and a control signal related to transmission and reception of ultrasonic waves, with the communication unit 201d included in the ultrasonic probe.

For example, the communication unit 113 receives periodical communication signals transmitted by the communication unit 201d and transmits reply signals thereto to the communication unit 201d. The communication unit 201d determines, by receiving the reply signals, whether the communication unit 201d is located within the range of communication with the apparatus body 100. The communication unit 113 then determines the connection through radio communication with the ultrasonic probe 201 including the communication unit 201d by receiving information of the connection through radio communication notified by the communication unit 201d. Upon receiving a pressed-down signal from the communication unit 201d, the communication unit 113 forwards the pressed-down signal thus received to the control circuit 112. The communication unit 113 transmits a control signal for controlling piezoelectric vibrators and a control signal for controlling the light emitting unit 201b to emit light to the communication unit 201d.

The following will describe an example of the processing performed by an ultrasound diagnostic apparatus 1 according to the third embodiment. The following description exemplifies a case of using the ultrasonic probe 201 with the color of a light emitting unit changed. For example, with the ultrasound diagnostic apparatus 1 transmitting and receiving a control signal through radio communication, the operator first brings the desired ultrasonic probe 201 into the range of communication with the apparatus body 100 to establish connection through radio communication.

The connection is thus established between the communication unit 201d and the communication unit 113, enabling transmission and reception of the control signal. This is the same state where the cable of the ultrasonic probe is connected to the probe interface unit 110. The control circuit 112 brings the light emitting unit 201b in the first light emission state by transmitting the first light emission signal to the ultrasonic probe 201 through the communication unit 113. For example, the light emitting unit 201b emits light in a predefined color based on the first light emission signal.

Thereafter, when the press-down button 201a is pressed down by the operator, the communication unit 201d transmits the press-down signal to the communication unit 113. The communication unit 113 transmits a control signal related to transmission and reception of ultrasonic waves corresponding to the press-down signal (a control signal for bringing the ultrasonic probe into the active state) and the second light emission signal, to the communication unit 201d. The communication unit 201d receives the control signal and the second light emission signal to bring the ultrasonic probe 201 into the active state and forwards the second light emission signal to the light emitting unit 201b to bring the light emitting unit 201b into the second light emission state. For example, the light emitting unit 201b emits light in a color different from the predefined color, based on the second light emission signal.

When the press-down button 201a is pressed down by the operator, the communication unit 201d transmits the press-down signal to the communication unit 113. The communication unit 113 transmits the control signal related to transmission and reception of ultrasonic waves corresponding to the press-down signal (the control signal for bringing the ultrasonic probe 201 into the freeze state) and the third light emission signal, to the communication unit 201d. The communication unit 201d receives the control signal and the third light emission signal to bring the ultrasonic probe 201 into the freeze state and forwards the third light emission signal to the light emitting unit 201b to change the light emission state of the light emitting unit 201b to the second light emission state. For example, the light emitting unit 201b emits light in a color different from the color based on the second light emission signal, based on the third light emission signal.

As described above, in a similar manner to the wired control, the control through radio communication can still transmit and receive the control signal related to transmission and reception of ultrasonic waves and the light emission signal between the ultrasonic probe 201 and the apparatus body 100, thereby consistently controlling the press-down button 201a and the light emitting unit 201b of the ultrasonic probe 201.

During the radio communication described above, when the ultrasonic probe is moved out of the range of communication with the apparatus body, the operator is notified that the ultrasonic probe is moved out of the range of communication. For example, the communication unit 201d determines that the ultrasonic probe 201 is moved out of the range of communication with the apparatus body 100, notifying the light emitting unit 201b of the result of the determination. When notified that the ultrasonic probe 201 is moved out of the range of communication with the apparatus body 100, the light emitting unit 201b changes the current lighting (or blinking) state of light emission to the turned-off state. Specifically, when the ultrasonic probe 201 is moved out of the range of communication, the light emitting unit 201b is turned off to notify the operator of that the ultrasonic probe 201 is moved out of the range of communication.

Although the example above describes a case where the light emitting unit 201b changes the light color, the embodiment is not limited thereto, but lighting, blinking, the intensity of the light, or the like may be changed, for example.

The structure of the ultrasonic probe illustrated in FIG. 10 is merely an example and the embodiment is not limited thereto. For example, the ultrasonic probe 201 may be provided with an additional switch for switching the power source on and off. In such a case, the operator holds in hand the ultrasonic probe 201 placed in a probe holder installed near the apparatus body 100 of the ultrasound diagnostic apparatus 1 and turns on the switch thereof to achieve connection through radio communication by the communication unit 201d. This reduces power consumption compared to a case where the communication unit 201d periodically transmits communication signals.

The first and second embodiments described above have described a case where the ultrasonic probe 201 includes the press-down button 201a, the light emitting unit 201b, and the model name 201c. The embodiments are, however, not limited thereto, but are also applicable to cases where only the press-down button 201a is included, where the press-down button 201a and the light emitting unit 201b are included, and where the press-down button 201a and the model name 201c are included.

The first and second embodiments described above have described cases where a press-down button is used as a receiving unit for receiving the switching operation of the ultrasonic probes. The embodiments are, however, not limited thereto, but a switch may be used, for example.

The positions of the press-down button, the light emitting unit, and the model name are not limited to the positions illustrated in FIG. 3, but can be any positions. For example, the positions may be on a side face of the ultrasonic probe 201.

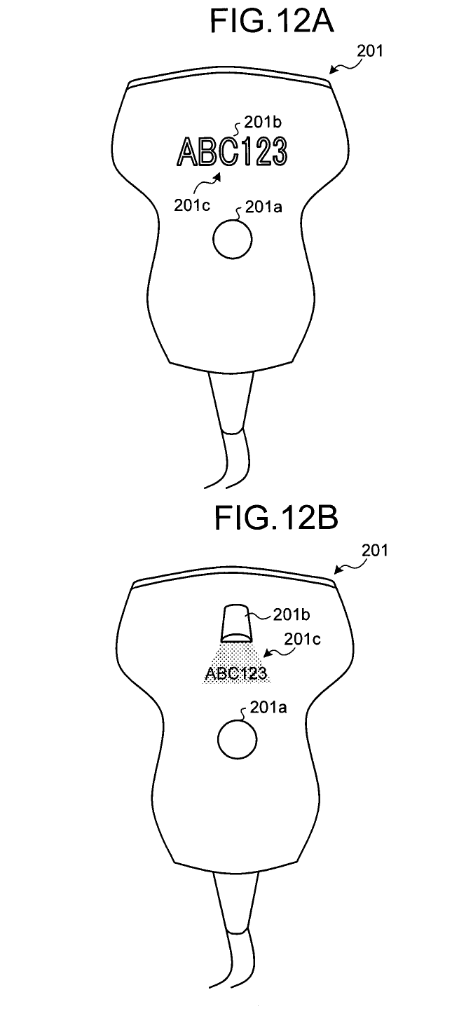

The first and second embodiments described above have described cases where the model name is printed on the front face of the light emitting unit 201b. The embodiments are, however, not limited thereto, but the position of the model name can be changed optionally. FIGS. 12A and 12B each are a diagram illustrating an example of the structure of the ultrasonic probe 201 according to the third embodiment. For example, in the ultrasonic probe 201, the transparent plastic material on the front face of the light emitting unit 201b may be formed with the model name 201c so that the model name 201c emits light as illustrated in FIG. 12A.

The light emitted by the light emitting unit 201b may be applied onto the model name 201c on the ultrasonic probe 201, for example, as illustrated in FIG. 12B.

The first and second embodiments described above have described cases where the model name of the ultrasonic probe is used as identification information for identifying the ultrasonic probe. The embodiments are, however, not limited thereto, but an identifier (a probe ID, for example) assigned to each ultrasonic probe may be used, for example.

With an ultrasonic probe according to at least one embodiment described above, diagnostic efficiency can be improved when a plurality of ultrasonic probes are switched over for use in diagnosis.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic probe having a piezoelectric vibrator transmitting and receiving ultrasonic waves under control of an apparatus body, the ultrasonic probe comprising:
a receiving unit configured to receive input operation related to transmission and reception of ultrasonic waves performed by the piezoelectric vibrator; and
a light emitting unit configured to emit light in a first light emission state when connected to the apparatus body and emit light in a second light emission state when the receiving unit receives input operation in a state that the light emitting unit is connected to the apparatus body.

2. The ultrasonic probe according to claim 1, wherein the light emitting unit emits light in a state that at least one of the color of the light, lighting, blinking, and the intensity of the light is changed between the first light emission state and the second light emission state.

3. The ultrasonic probe according to claim 1, wherein the light emitting unit emits light while changing light emission state as the second light emission state each time the receiving unit receives input operation in a state that the light emitting unit is in the second light emission state.

4. The ultrasonic probe according to claim 1, wherein the light emitting unit is disposed in a position at the side of the piezoelectric vibrator relative to the receiving unit on the ultrasonic probe.

5. The ultrasonic probe according to claim 1, further comprising identification information for identifying the ultrasonic probe, wherein
the identification information is irradiated with light emitted by the light emitting unit.

6. The ultrasonic probe according to claim 1, further comprising a communication unit performing transmission and reception of a control signal with the apparatus body through a radio signal, wherein
the communication unit transmits a radio signal corresponding to input operation received by the receiving unit to the apparatus body.

7. An ultrasound diagnostic apparatus comprising:
the ultrasonic probe according to any one of claims 1 to 6;
a connector for connecting the ultrasonic probe to the apparatus body;
a transmitter-receiver unit configured to control transmission and reception of ultrasonic waves;
a controller configured to control the transmitter-receiver unit to cause the ultrasonic probe to perform transmission and reception of ultrasonic waves when the receiving unit included in the ultrasonic probe receives input operation related to transmission and reception of ultrasonic waves in a state that the ultrasonic probe is connected to the apparatus body through the connector; and
a light emitting controller configured to control the light emitting unit included in the ultrasonic probe to emit light in the first light emission state when the ultrasonic probe is connected to the apparatus body through the connector and control the light emitting unit to emit light in the second light emission state when the receiving unit receives input operation in a state that the ultrasonic probe is connected to the apparatus body through the connector.
